(19) United States
(12) Patent Application Publication
(10) Pub. No.: US 2022/0331019 A1
(43) Pub. Date: Oct. 20, 2022

(54) MULTIMEDIA AND VIRTUAL REALITY PATIENT SAFETY SYSTEM FOR MEDICAL AND SURGICAL PROCEDURES

(71) Applicant: WIDE AWAKE VR INC, Southfield, MI (US)

(72) Inventors: Joseph W Gough, Whitmore Lake, MI (US); Cecil David Wheeler, Palos Verdes Peninsula, CA (US)

(73) Assignee: WIDE AWAKE VR INC, Southfield, MI (US)

(21) Appl. No.: 17/763,559

(22) PCT Filed: Feb. 16, 2021
(86) PCT No.: PCT/US2021/018186

§ 371 Date: Mar. 24, 2022

Related U.S. Application Data
(60) Provisional application No. 62/977,470, filed on Feb. 17, 2020.

(51) Int. Cl. A61B 34/00; A61B 90/50; G06N 20/00

(52) U.S. Cl. CPC A61B 34/25; A61B 90/50; G06N 20/00; A61B 2090/502; A61B 2034/258

(57) ABSTRACT

A patient safety system and method for delivering immersive multimedia and virtual reality content in a clinically safe and secure manner. The system and method for matching personalized virtual reality content with a user profile in a clinically safe and secure manner for the purpose of creating externally controlled immersive experiences during surgical procedures or medical treatments. Our invention takes personality type, anxiety levels, medical condition, procedural orientation, physical limitations, surgical complexity, duration of surgery, and content preference into account. Then it presents the content in a viewable headset interface that can be externally controlled by the healthcare provider for the safety of the patient. Both audio and visual communication are available between the healthcare provider and the patient to make the surgical procedure efficient for the healthcare provider and comfortable for the patient. Finally, the system matches preoperative and postsurgical content for the patient's benefit in preparation and recovery from the surgery.

### CROSS REFERENCE TO RELATED APPLICATIONS

This application claims the benefit to U.S. Provisional Application No. 62/977,470 filed on Feb. 17, 2020, the entire contents of which are incorporated herein by reference.

### FIELD OF THE INVENTION

A system for matching personalized virtual reality content with a user profile in a clinically safe and secure manner for the purpose of creating externally controlled immersive experiences during surgical procedures or medical treatments.

### BACKGROUND OF THE INVENTION

Many patients experience anxiety during medical procedures, from complicated surgeries to simple injections. Patient experiences would be better and health procedure costs lower if patients were calm during treatment, allowing healthcare providers to confidently use local anesthesia instead of general anesthesia for minor surgeries, and in some cases such as simple injections, use no anesthesia at all. The problem is that no solution exists besides anesthesia to safely help a patient who is anxious or stressed about a medical procedure. Additionally, there is currently no process at all in which the patient can become involved in the procedure, be in contact with the doctor, be prompted to react to doctor's instructions, and at the same time become distracted and remain calm during a medical procedure or treatment.

The present invention determines personality type, anxiety levels, medical condition, procedural orientation, physical limitations, surgical complexity, and duration of surgery to take into account the unique personality and preferences of the patient. The invention presents the content in a viewable interface that can be externally controlled by the healthcare professional for the safety of the patient. Finally, the system matches pre- and post-surgical content for the unique patient's preparation and recovery.

### SUMMARY OF THE INVENTION

A wide-awake virtual reality (VR) procedure and system for creating a controlled immersive experience for a subject undergoing a surgical procedure or medical treatment by a healthcare provider comprising the steps:

a) the healthcare provider enrolls the patient in the program using the software program's provider interface, which triggers a message to the patient requiring their confirmation and further input;

b) the patient electronically consents to the wide-awake VR procedure and assessment of available patient health records via a software program and patient user interface;

c) the patient's personality type and preferences are acquired through an interactive digital survey program, which alerts the provider that consent, and patient data are ready for review;

d) the healthcare provider enters the surgical procedure information or medical treatment information into the provider portal of the software program;

e) all the data acquired via electronic health records and reported directly by patients and healthcare providers is transferred into an adaptive or machine learning algorithm for patient matching, satisfaction, and safety optimization scoring;

f) the resulting score is used to match the patient to a scored and tagged curated VR content grouping stored on a database server;

g) the database server receives a message to send the tagged curated VR content grouping to the provider portal for approval;

h) following approval by the healthcare provider of the tagged curated VR content grouping, the patient selects a specific content grouping for viewing during the upcoming surgical procedure or medical treatment;

i) the patient-selected VR content is sent to the provider portal for use during the surgical procedure or medical treatment;

j) the patient is fitted with a VR headset user interface capable of displaying visual and audio content during the surgical procedure or medical treatment;

k) following the fitting of the VR headset user interface, the selected VR content is pre-loaded to a content menu to allow the patient to access the content in the VR headset user interface to provide an immersive and personalized VR experience during the surgical procedure or medical treatment; and

l) at the conclusion of the surgical procedure or medical treatment, the healthcare provider sends a message to the database server to end the wide-awake VR immersive procedure.

### DETAILED DESCRIPTION OF THE INVENTION

### Definitions

Virtual Reality—Virtual Reality (VR) is an interactive computer-generated experience that allows users to react as if it were a real, physical, environment. The human brain actively models the world using sensory information. By replacing auditory, visual, and even tactile and olfactory stimuli from the physical world with digital input, the user (in this case the patient) becomes involved in the virtual reality and distracted from the ongoing medical procedure while remaining awake rather than partially or completely sedated.

Augmented Reality—AR is an interactive experience of a real-world environment where the objects that reside in the real world are enhanced by computer-generated perceptual information, sometimes across multiple sensory modalities, including visual, auditory, haptic, somatosensory, and olfactory.

Teletherapy—also known as online therapy, e-therapy, or video therapy—is therapy provided by a healthcare provider delivered through a virtual platform via a computer.

Machine Learning System—Our platform uses multiple adaptive algorithms to determine appropriate content for the patient. The system is trained through the manual selection of meta-tagged content groupings. Every time a selection is chosen by a patient and approved by the surgeon for a specific procedure, that selection becomes more qualified for that procedure and that personality type. The selection can become more qualified when a postoperative survey shows high satisfaction from both the patient's and surgeon's perspectives. Likewise, non-selections and poor survey reviews will disqualify that content for future use under the procedure and personality circumstances that coincide with “non-selection” or “poor review.”

Throughout the description and claims of this specification, the word “comprise,” (and variations of the word, such as “comprising” and “comprises,”) means “including but not limited to,” and is not intended to exclude, for example, other additives, components, integers, or steps.

The phrase “and/or” as used herein, should be understood to mean “either or both” of the elements so conjoined, i.e., elements that are conjunctively present in some cases and disjunctively present in other cases.

As used herein, “or” should be understood to have the same meaning as defined above for “and/or”. For example, when separating a listing of items, “and/or” or “or” shall be interpreted as being inclusive, i.e., the inclusion of at least one, but also including more than one, of a number of items, and, optionally, additional unlisted items. Only terms clearly indicated to the contrary, such as “only one of” or “exactly one of”, or, when used in the claims, “consisting of”, will refer to the inclusion of exactly one element of a list of elements. In general, the term “or” as used herein shall only be interpreted as indicating exclusive alternatives (i.e., “one or the other but not both”) when preceded by terms of exclusivity, such as “either”, “one of”, “only one of”, or “exactly one of.”

As stated above, many patients experience anxiety during medical procedures, from complicated surgeries to simple injections. Patient experiences would be better and health procedure costs lower if patients were calm during treatment, allowing healthcare providers to confidently use local anesthesia instead of general anesthesia for minor surgeries, and in some cases such as simple injections, use no anesthesia at all. The problem is that no solution exists besides anesthesia to safely help a patient who is anxious or stressed about a medical procedure. Additionally, there is currently no process at all in which the patient can become involved in the procedure, be in contact with the doctor, be prompted to react to doctor's instructions, and at the same time become distracted and remain calm during a medical procedure or treatment.

Wide-Awake VR procedure matches the perfect virtual reality content to both the personality of the user and the safety factors of the procedure. By combining health records, survey tools, and diagnostic measures, a personalized and immersive experience is provided and administered to the patient by the doctor. The system goes further by offering to patient's preoperative experiences so they can prepare for what to expect, and doctors and nurses are provided with live surgical controls in order to communicate inside the virtual reality experience as well as postoperative therapy planning in order to facilitate the safest patient recovery after treatment or surgery.

The claimed invention differs from technology that currently exists. Current virtual reality systems do not have data analytics programs, survey tools, algorithms, or data entry portals to gather and assess the most immersive and clinically safe content for each person and procedure. More important, no system exists to allow for the chosen immersive experiences to be externally controlled by the healthcare provider, or with multi-channel communication capabilities between patient and provider.

This invention is an improvement on what currently exists because the safety of the patient is the top priority of every surgical episode. If healthcare providers cannot safely and responsibly control the content of the VR experience, they risk patient agitation, disorientation, or over-sedation during key moments of the surgery. Furthermore, the personalized content matching for virtual reality experiences as well as the preoperative and postoperative instructions are vital to the overall success of the surgical episode and patient recovery. Neither of these features existed before the present Wide-Awake VR procedure system was developed.

This invention takes into account a patient's personality type, anxiety levels, medical condition, demographics, content preference, and physical limitations as well as surgical complexity, duration of surgery, and procedural orientation. Then it presents the content in a viewable interface that can be externally controlled by the healthcare professional for the safety of the patient.

The invention can produce the reporting necessary to drive value-based care policy changes, patient confidence, and patient satisfaction that will increase provider reimbursement in alternative payment models. Likewise, the invention will produce reporting and patient outcomes that encourage transformative incentive structures for health insurance plans and government programs trying to lower the cost of unnecessary medical procedures.

Currently available VR systems are set up for the purpose of entertainment, not medical treatment. Any preference algorithms are built for personal taste, not personality type or medical conditions. Today's VR systems are designed for subjects to be in control of their own experience. In clinical practice, it is more beneficial for the healthcare provider to control the system for the safety of the patient.

### Wide-Awake Virtual Reality Procedure

A wide-awake virtual reality (VR) procedure for creating a controlled immersive experience for a patient undergoing a surgical procedure or medical treatment by a healthcare provider comprising the steps.

Step 1. The healthcare provider enrolls the patient in the program using the software program's provider interface, which triggers a message to the patient requiring their confirmation and further input.
Step 2. Patient electronically consents to Wide-Awake VR procedure and assessment of available personal health records via a software program and patient user interface.
Step 3. Patient's personality type and preferences are acquired through an interactive digital survey program, which alerts the provider that consent, and patient data are ready for review.
Step 4. The healthcare providers enter the surgical procedural information or medical treatment information into the provider portal of the software program.
Step 5. The data inputs from the patient's health records, survey results, and procedural information are transferred into an adaptive or machine learning algorithm for patient matching, satisfaction, and safety optimization.
Step 6. The resulting score produced by the algorithm matches the patient to a curated content grouping stored in a database.
Step 7. The database server receives a call to send the matching curated content group to the provider portal for review and approval.
Step 8. Once approved by the provider, the patient chooses from the approved curated content group that they wish to view during the procedure, which is sent to the provider portal for use during the procedure.
Step 9. The patient is automatically assigned preoperative content via secure messaging.
Step 10. As the procedure begins, the patient is fitted with a VR headset and earphones viewer (VR content interface). The immersive and personalized virtual reality content chosen plays inside the patient's viewer headset and earphone while the procedure being done.
Step 11. Healthcare providers can externally control the content and communicate directly with the patient by talking through the audio system and/or through text messaging into the VR content interface.
Step 12. Upon completion of the procedure, the healthcare provider can automatically send the patient postoperative treatment content via secure messaging to achieve optimal recovery.

### Relationship Between Wide-Awake Virtual Reality Procedure Components

The invention functions as a multiple component system with a centralized database and three user interfaces; the patient portal, the physician portal, and the VR content interface. All of these are deployed from the same integrated software platform. The procedure begins when the healthcare provider enrolls the patient in the program using the software program's provider interface, which triggers a message to the patient requiring their confirmation and further input (Step 1).

The next step in the process is the patient giving electronic consent and enrolling in the Wide-Awake VR procedure through an interactive user interface on a computer or mobile device (step 2). The enrollment and consent submission triggers a call to the database server for the collection of additional data through application programming interfaces (APIs) allowing data exchange with electronic health record systems or health information exchanges (Step 2).

After patient enrollment, a software interface presents a survey to gather the personality type and preferences of the patient (Step 3).

With the patient's information complete, a communication goes to the server which triggers the provider portal. The healthcare professionals handling the procedure receive a multi-channel notification via email, text, or push notice. This message tells the physician and their staff that they have a patient in the queue. The provider must provide a second interface in order to enter the procedural information required to complete the necessary episode information (Step 4).

A data package of procedural information, patient-reported information, and health records are then sent to the database and entered into an adaptive or machine learning algorithm for scoring (Step 5). The score calculated by the algorithm determines the matching content experiences. The database produces a content group of approved virtual reality experiences that match the needs and preferences of the patient's procedure and personality (Step 6).

Once the content group is identified, it is automatically sent to the provider portal. From there, the provider must review and approve the content group for the patient experience and that approval triggers a notification to the patient for a final selection of content (Step 7).

When the patient makes their selection, a communication goes back to the database and preoperative content is selected and sent to the patient via multi-channel notification (Step 8).

When the patient arrives at the doctor's office for their procedure, they are fitted with a standard VR headset comprising earphones, a viewable screen, and a controller. The headsets are designed for virtual reality content viewing. The provider enters their name in the provider portal and the headset viewing interface opens a menu with the preloaded content of their choice. The content can be launched by the patient or the provider. The provider sees the content being viewed in their connected external monitor and has control to override the program (Step 9).

During the procedure, the healthcare providers can communicate with the patient via text in a chat block on the viewing interface inside the headset. The doctor can also communicate by overriding the audio and using an external microphone to speak directly to the patient. The providers can use their external controls to stop, start, pause, or reset content at any time. This gives them the control required to guarantee patient safety throughout the surgical episode (Steps 10 and 11).

Upon completion of the procedure, the provider triggers a final data submission in which time duration, content viewed, and patient records are packaged for reporting purposes and sent to the database. The database holds postoperative content that can optionally automatically be sent to the patient through a multi-channel notification after the completion data is received (Step 12).

### How the Wide-Awake Virtual Reality Procedure Works

The system provides a novel and unique solution to patient anxiety problems that plague healthcare providers who feel they must use general anesthetic for certain procedures on certain patients. The system enables the safer and more cost-effective use of local anesthetic for numerous minor surgeries and medical procedures in simple procedures such as injections and infusions. The digital interfaces of the system collect enrollment information, demographics, survey answers, and procedural information necessary to customize the virtual reality content. The database and the machine learning program hold the repository of organized virtual reality content, and by using the data entered to create a score and match that score with the qualified content.

The servers and the software programs allow for the logistical entering, routing, and transfer of the data required to successfully complete the process. The computing hardware comprising monitors, keyboards, VR headsets, and tablets or laptops allow both data entry and presentation of content to be facilitated in the appropriate environments. The virtual reality hardware allows patients to view the content in an immersive manner where they are comfortable. All the while, the healthcare provider remains in control of the experience through an external monitor, microphone, controller, mouse and/or keyboard. In combination, these components create a unique benefit for patients, providers, and payers in the healthcare ecosystem.

The patient and provider experiences may be described in the following manner: If the patient consents to use Wide-Awake VR, then the application programming interface call for health records is triggered and the content for the patient survey is loaded from the database server. If the patient does not consent, or has a question about their consent, the provider is notified for a consult.

If the patient completes the survey effectively, the provider is notified to enter the portal and provide the procedural information. If the patient does not complete the survey, the provider is notified for a consult.

If the provider enters the procedural data effectively, then the combined data package is sent to the database and is processed into the machine learning system for content scoring. If something in the data package is missing or incomplete, the patient or the provider would be notified of the missing information.

Depending on a multi-variable score, which factors weighted integers in several categories and matches personalized content, is processed in the database program and the best content for the patient and procedure is selected. The scoring logic takes the procedural information into priority to account first for ensure patient safety. The curation process then moves to health records (if available) to detect other potential health and safety factors. Finally, the patient's personality and preferences are weighted equally for entertainment and attention value, only after all safety precautions have been calculated and prioritized.

If matching content exists, it is sent to the provider for approval. If no matching content can be safely featured, a notification is sent to Wide-Awake VR's content administration team, so that they can manually enter the most effective content for the procedure.

If the provider approves the content grouping, it is sent to the patient for final preference selection. If the provider does not approve, a message is sent to the Wide-Awake VR content administration, so that they can manually select content that the provider deems more appropriate.

When the final content selection is made, it is transferred from the database to the menu program which appears in the VR headset viewing area prior to surgery.

The patient is fitted with the headset when arriving in the surgical suite. After the healthcare providers prepare the patient, the patient is allowed to select their content from the start menu to begin the content experience.

Throughout the experience, the providers monitor the mirrored content in their external monitor. The providers have complete control over the content at all times. They can stop, start, pause, and interrupt content with audio or visual messaging that they deem necessary to effectively communicate with the patient.

At the conclusion of the surgery, the provider closes the system and triggers the sending of postoperative content to the patient via email. This content helps guide the patient through their recovery in the most responsible manner.

### How to Make the Wide-Awake Virtual Reality Procedure System

The virtual reality procedure system comprises a new form of computing interface for virtual reality programs, one that allows for the external control of the content and communication from an outside party within the virtual reality viewing experience. This platform requires a mirrored viewing experience, external controllers, audio/visual intervention controls, and the software to manage all the functional commands demanded within the virtual reality experience. With this innovation, the inventor can program a machine learning program that calculates and scores inputs from a variety of sources and match the resulting data score to meta-tagged content stored in a database. The interaction between user interfaces, machine learning algorithms, virtual reality content libraries, and the new system of virtual reality presentation with external control programming and hardware combine to create the complete Wide Awake VR procedure system.

In order to operate the invention successfully, the system requires a computing device that allows data to be entered directly to a physical server or transferred via API calls to a cloud-based server. The invention further requires a machine learning software program that applies artificial intelligence to received data, calculates a score, and matches that score to correlated content packages stored in the connected database. Finally, the virtual reality hardware and software components work in coordination to surface the content in the headset viewing display and allow for user manipulation from the content menu, while also allowing the healthcare provider to govern the experience and communicate with the patient. This is accomplished by a new virtual reality procedure system that gives external override capabilities to the healthcare professional. By using a mirrored content monitor, the provider can see what the patient is seeing. By using a keyboard or natural language voice processor, the providers can communicate with the patient visually in the display field of the headset or audibly through a microphone and headphone combination. By using the software program, the provider can stop, start, or change the content being viewed at any time to achieve the safe and secure success of the patient experience.

The optional elements are the various types of interfaces and computing devices used in the data entry phase of the process. Any type of device and any type of interface can be used to enter the data required to begin the machine learning process of the system. Likewise, the headsets, monitors, controllers, and display hardware are all optional elements.

### How to Use the Wide-Awake Virtual Reality Procedure

People that have complex health conditions should not undergo surgery using unnecessary general anesthesia. This invention will allow many surgical procedures in which healthcare providers currently use general anesthesia, because of patient anxiety or discomfort, to be conducted under local anesthetic. Even patients suffering from anxiety over needles, infusions, or other simple invasive procedures (such as shots) should have viable, safe, non-narcotic solutions offered to them for their anxiety. The immersive deployment of Wide-Awake VR allows for both simple patient anxiety and healthcare surgical anesthetic to be optimized for the safety and comfort of the patient.

The invention can also produce the reporting necessary to drive value-based care policy changes, patient confidence, and patient satisfaction reporting that will increase provider reimbursement in alternative payment models. The invention will produce patient outcome reporting that encourages transformative new payment models for health insurance plans and government payers.

1. A wide-awake virtual reality (VR) procedure for creating a controlled immersive experience for a patient undergoing a surgical procedure or medical treatment by a healthcare provider comprising the steps:
a) the healthcare provider enrolls the patient in the program using the software program's provider interface, which triggers a message to the patient requiring their confirmation and further input;
b) the patient electronically consents to the wide-awake VR procedure and assessment of available patient health records via a software program and patient user interface;
c) the patient's personality type and preferences are acquired through an interactive digital survey program, which alerts the provider that consent, and patient data are ready for review;
d) the healthcare provider enters the surgical procedure information or medical treatment information into the provider portal of the software program;
e) all the data acquired via electronic health records and reported directly by patients and healthcare providers is transferred into an adaptive or machine learning algorithm for patient matching, satisfaction, and safety optimization scoring;
f) the resulting score is used to match the patient to a scored and tagged curated VR content grouping stored on a database server;
g) the database server receives a message to send the tagged curated VR content grouping to the provider portal for approval;
h) following approval by the healthcare provider of the tagged curated VR content grouping, the patient selects a specific content grouping for viewing during the upcoming surgical procedure or medical treatment;
i) the patient-selected VR content is sent to the provider portal for use during the surgical procedure or medical treatment;
j) the patient is fitted with a VR headset user interface capable of displaying visual and audio content during the surgical procedure or medical treatment;
k) following the fitting of the VR headset user interface, the selected VR content is pre-loaded to a content menu to allow the patient to access the content in the VR headset user interface to provide an immersive and personalized VR experience during the surgical procedure or medical treatment; and
l) at the conclusion of the surgical procedure or medical treatment, the healthcare provider sends a message to the database server to end the wide-awake VR immersive procedure.

2. The wide-awake VR procedure of claim 1, wherein prior to step j) the patient is automatically assigned and sent correlated content related to the surgical procedure or medical treatment.

3. The wide-awake VR procedure of claim 1, wherein the content viewed by the patient during the surgical procedure or medical treatment is mirrored for external viewing on a monitor positioned by the healthcare provider.

4. The wide-awake VR procedure of claim 1, wherein a software program and/or controllers allow the healthcare provider to have complete external control of the selected VR content viewed by the patient.

5. The wide-awake VR procedure of claim 4, wherein the software program and/or controllers are configured to allow for stop, start, reverse, fast-forward, and volume control of the selected VR content viewed by the patient in the VR headset user interface.

6. The wide-awake VR procedure of claim 1, wherein a software program, headset, controllers and/or microphones allow the healthcare provider to communicate with the patient through the VR headset user interface.

7. (canceled)

8. The wide-awake VR procedure of claim 6, wherein the communication allows the healthcare provider to direct the patient to gauge the patient's range of motion and other important physical actions.

9. The wide-awake VR procedure of claim 1, wherein in the VR headset user interface a graphic progress bar or clock timer is displayed in the patient selected VR content allowing the patient to monitor the progress of the surgical procedure or medical treatment.

10. The wide-awake VR procedure of claim 1, wherein at the completion of the surgical procedure or medical treatment, the healthcare provider selects from the database software custom multimedia discharge instructions related to the surgical procedure or medical treatment for viewing by the patient in the VR headset or in a traditional media display.

11. The wide-awake VR procedure of claim 10, wherein the patient is automatically through the database server provided post-operative content and discharge instructions related to continuing recovery from the surgical procedure or medical treatment.

12. The wide-awake VR procedure of claim 1, wherein the patient upon discharge by the healthcare provider is provided with VR headset user interface for use in outpatient follow-up recovery therapy.

13. The wide-awake VR procedure of claim 12, wherein the outpatient follow-up recovery therapy is provided by teletherapy or by recorded VR content for asynchronous viewing.

14. The wide-awake VR procedure of claim 3, wherein during the surgical procedure or medical treatment brain activity is monitored along with the mirrored VR content to allow the healthcare provider to control the VR content to enhance the patient experience.

15. A system for creating a customized wide-awake virtual reality (VR) immersive experience in a patient during a surgical procedure or medical treatment performed by a healthcare provider comprising:
a) a VR headset for fitting on the patient, which is capable of displaying customized wide-awake VR immersive content;
b) a centralized database server with an integrated software platform accessible by a patient portal interface, a healthcare provider portal interface, and a VR headset portal interface;
c) a software program and data in the integrated software platform accessible by the patient through the patient portal interface, which comprises information on the customized wide-awake VR immersive experience content and the surgical procedure or medical treatment to be performed by the healthcare provider and a patient consent and enrollment form to allow the healthcare provider to conduct the surgical procedure or medical treatment using and controlling the customized wide-awake VR immersive experience;
d) a software program that communicates with the integrated software platform for collecting enrollment information, demographics, and personality type and preferences provided by patient survey answers;
e) a software program in the integrated software platform that is automatically triggered by patient consent and enrollment to message the centralized database server to collect patient healthcare data through application programming interfaces that allow for data exchange with electronic health record systems or health information exchanges;
f) a software program in the integrated software platform that communicates with the healthcare provider through a multi-channel notification comprising email, text, or push messaging, following receipt of an input that the patient has given consent and enrolled to have the healthcare provider perform the surgical procedure or medical treatment using and controlling the customized wide-awake VR immersive experience content;
g) a software program in the integrated software platform that is accessed by the healthcare provider through the healthcare provider portal interface that allows the healthcare provider to input the surgical procedure or medical treatment;
h) an algorithm for scoring the input patient demographic information, personality type and preferences survey answers, patient healthcare data, and surgical procedure or medical treatment information;
i) a first algorithm for scoring and tagging a catalog of a wide-awake VR immersive experience content stored on the centralized database server;
j) a second algorithm that matches the score and tags for the input patient demographic information, personality type and preferences survey answers, patient healthcare data, and surgical procedure or medical treatment information to the scored and tagged catalog of wide-awake VR immersive experience content, wherein the second algorithm provides a matched grouping of wide-awake VR immersive experience content from the catalog for review and approval by the healthcare provider;
k) a software program in the integrated software platform that sends a multi-channel notification comprising email, text, or push messaging to the patient that the healthcare provider approved group of wide-awake VR immersive experience content is available for the patient to select the specific wide-awake VR immersive experience for the surgical procedure or medical treatment; and
l) a software program in the integrated software platform that allows the selected customized wide-awake VR immersive experience content to be accessible from a menu program in the VR headset by the patient during the surgical procedure or medical treatment, wherein the system provides a controlled customized wide-awake virtual reality (VR) immersive experience for a patient during the surgical procedure or medical treatment.

16. The system of claim 15, wherein the first algorithm in i) is an adaptive or machine learning algorithm.

17. The system of claim 15, wherein the second algorithm in j) is an adaptive or machine learning algorithm.

18. The system of claim 15, further comprising a monitor for mirrored viewing by the healthcare provider of the selected customized wide-awake immersive experience during the surgical procedure or medical treatment.

19. The system of claim 15, further comprising a software program, microphone, integrated headset and/or controller that allows the healthcare provider to communicate directly with the patient in the VR headset.

20. The system of claim 15, further comprising a software program and/or controller that allows the healthcare provider to stop, start, and/or change the wide-awake VR immersive experience content being viewed at any time during the surgical procedure or medical treatment.

21. A wide-awake virtual reality (VR) procedure for creating a controlled immersive experience for a patient undergoing a surgical procedure or medical treatment by a healthcare provider comprising the steps:
a) selection of a grouping of wide-awake VR immersive content for review and approval by the healthcare provider by matching an adaptive or machine learning algorithm scored and tagged input patient demographic information, patient personality type and preferences survey answers, patient healthcare data, and surgical procedure or medical treatment information to a scored and tagged catalog of wide-awake VR immersive content;
b) sending to the patient a multi-channel notification comprising email, text, or push messaging that the healthcare provider reviewed and approved grouping of wide-awake VR immersive content is available for the patient to select the specific wide-awake VR immersive content they wish to view during the surgical procedure or medical treatment;
c) a VR headset user interface is fitted on the patient, which is capable of displaying the patient selected wide-awake VR immersive content;
d) the selected wide-awake VR immersive content is made accessible to the VR headset user interface to allow the patient to play the patient selected wide-awake VR immersive content during the surgical procedure or medical treatment; and
e) during the surgical procedure or medical treatment the healthcare provider to has complete external control through of the selected VR immersive content viewed by the patient through a software program, controller, keyboard, and/or microphones.
